(19) United States
(12) Patent Application Publication
(10) Pub. No.: US 2012/0004499 A1
(43) Pub. Date: Jan. 5, 2012

(54) Surgical Method for Performing a Coronary Blood Vessel Bypass

(75) Inventor: Douglas E. Ott, Macon, GA (US)

(73) Assignee: Lexion Medical, LLC, St. Paul, MN (US)

(21) Appl. No.: 12/829,072

(22) Filed: Jul. 1, 2010

(51) Int. Cl. A61F 2/04; A61M 37/00

(52) U.S. Cl. 600/36; 604/24

(57) ABSTRACT

In accordance with one embodiment of the present disclosure, a surgical method may include making a first incision in a patient. The surgical method may also include receiving a gas from a source and humidifying and warming the gas received from the source. The surgical method may further include delivering the humidified and warmed gas into the first incision. The surgical method may further include separating one or more blood vessel branches from a blood vessel using at least one surgical instrument inserted through a second incision in the patient. The surgical method may further include removing a blood vessel segment from the patient through a third incision in the patient, wherein the blood vessel segment was in contact with the humidified and warmed gas delivered into the first incision prior to the removal.

### TECHNICAL FIELD

This disclosure relates in general to surgical operations and more particularly to a surgical method for performing a coronary blood vessel bypass.

### BACKGROUND

Traditional methods for performing a coronary blood vessel bypass involve removing a blood vessel from a patient and using that blood vessel to bypass a blockage in a coronary blood vessel. Such traditional methods, however, are deficient.

### SUMMARY OF THE DISCLOSURE

In accordance with one embodiment of the present disclosure, a surgical method may include making a first incision in a patient. The surgical method may also include receiving a gas from a source and humidifying and warming the gas received from the source. The surgical method may further include delivering the humidified and warmed gas into the first incision. The surgical method may further include separating one or more blood vessel branches from a blood vessel using at least one surgical instrument inserted through a second incision in the patient. The surgical method may further include removing a blood vessel segment from the patient through a third incision in the patient, wherein the blood vessel segment was in contact with the humidified and warmed gas delivered into the first incision prior to the removal.

Numerous technical advantages are provided according to various embodiments of the present disclosure. Particular embodiments of the disclosure may exhibit none, some, or all of the following advantages depending on the implementation. In certain embodiments, by removing a blood vessel segment that has been in contact with a humidified and warmed gas, the blood vessel segment may be in a better condition for use in a blood vessel bypass. For example, the humidified and warmed gas may reduce desiccation of the outside of the blood vessel segment, causing it to be more moist and pliable. Furthermore the humidified and warmed gas has the potential to reduce the inflammatory response of the blood vessel segment, reduce vasospasm in the blood vessel segment, reduce radial circumferential constriction in the blood vessel segment, reduce longitudinal contraction and shortening in the blood vessel segment, reduce adventitial damage in the blood vessel segment, and/or reduce outside vessel wall damage in the blood vessel segment.

Other technical advantages of the present disclosure will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present disclosure and its advantages, reference is now made to the following description, taken in conjunction with the accompanying drawings, in which:

FIG. 1 illustrates one embodiment of a surgical method for performing a coronary blood vessel bypass;

FIG. 2 is a schematic view of one embodiment of an apparatus that may be used in a surgical method;

FIG. 3 is a cross-sectional view of one embodiment of a heater/humidifier of an apparatus that may be used in a surgical method;

FIG. 4 is a schematic diagram of one embodiment of a heating element of an apparatus that may be used in a surgical method;

FIG. 5 is a cross-sectional view of one embodiment of a heater/humidifier of an apparatus that may be used in a surgical method;

FIG. 6 is a schematic diagram showing one embodiment of a control circuit of an apparatus that may be used in a surgical method; and

FIG. 7 is a schematic diagram showing a further embodiment of a control circuit of an apparatus that may be used in a surgical method.

### DETAILED DESCRIPTION OF THE DRAWINGS

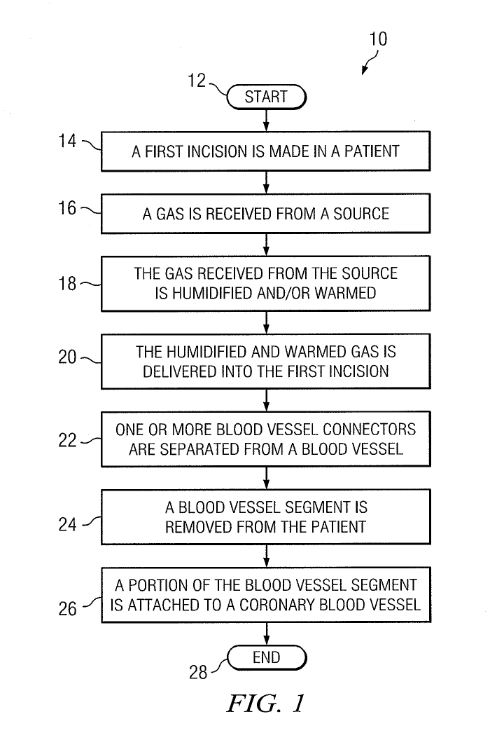

Embodiments of the present disclosure and their advantages are best understood by referring to FIGS. 1 through 7 of the drawings, like numerals being used for like and corresponding parts of the various drawings.

FIG. 1 illustrates one embodiment of a surgical method for performing a coronary blood vessel bypass. In one embodiment, the surgical method 10 may include delivering a humidified and warmed gas into a first incision of a patient, and removing a blood vessel segment from the patient. In particular embodiments, by removing a blood vessel segment that has been in contact with a humidified and warmed gas, the blood vessel segment may be in a better condition for use in a coronary blood vessel bypass. For example, the humidified and warmed gas may reduce desiccation of the outside of the blood vessel segment, causing it to be more moist and pliable. Furthermore the humidified and warmed gas has the potential to reduce the inflammatory response of the blood vessel segment, reduce vasospasm in the blood vessel segment, reduce radial circumferential constriction in the blood vessel segment, reduce longitudinal contraction and shortening in the blood vessel segment, reduce adventitial damage in the blood vessel segment, and/or reduce outside vessel wall damage in the blood vessel segment.

The method begins at step 12. At step 14, a first incision is made in a patient. In one embodiment, the patient may include a human being. In another embodiment, the patient may include an animal, such as a dog, cat, horse, pig, or any other suitable animal. In particular embodiments, the first incision is made in a limb of the patient. For example, the first incision may be made in a leg or an arm of the patient. The first incision in the limb of the patient may include an incision made in any area of the patient in order to access a portion of the limb of a patient. For example, the first incision in the limb of the patient may include an incision made in the upper body of the patient in order to access a blood vessel in the arm of the patient. As another example, the first incision made in the limb of the patient may include an incision in the lower body of the patient in order to access a blood vessel in the leg of the patient.

In one embodiment, the first incision may be made in the patient in order to access a blood vessel in the patient. For example, the first incision may be made in order to access a saphenous vein located in the leg of the patient. As another example, the first incision may be made in order to access a radial artery located in the arm of the patient. The first incision may be made in order to access any other suitable blood vessel. The first incision may be made with any suitable instrument. For example, the first incision may be made with a scalpel, knife, needle, or any other suitable instrument.

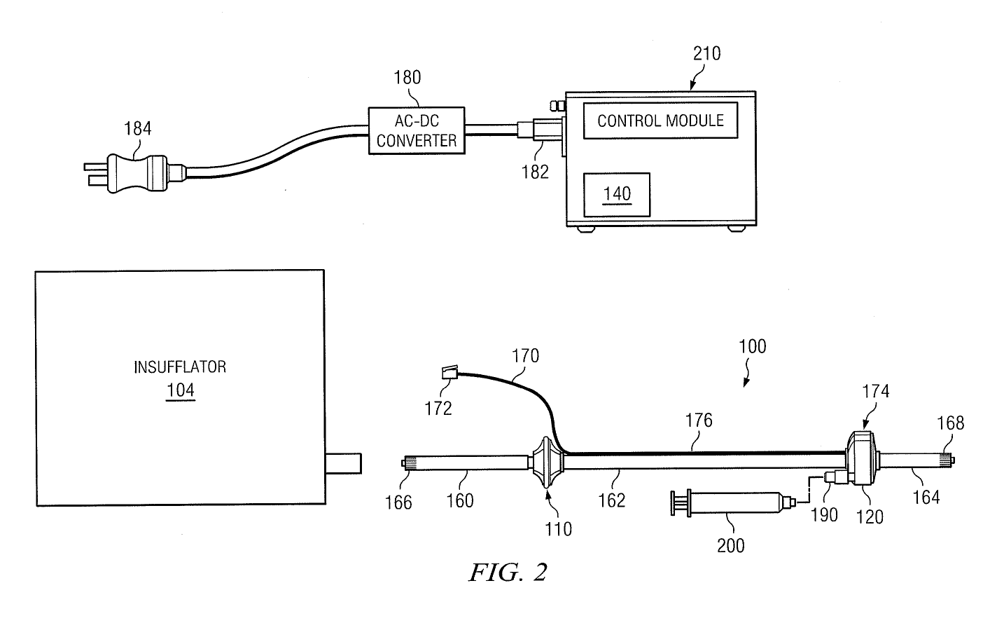

At step 16, a gas is received from a source. The gas may include any suitable gas. For example, the gas may include carbon dioxide, oxygen, nitrous oxide, argon, helium, nitrogen, room air, or other inert gases. In a further embodiment, the gas may include a combination of gases. For example, the gas may include a combination of carbon dioxide and nitrous oxide. In particular embodiments, the gas may be received at any suitable apparatus. For example, in one embodiment, the gas may be received at the apparatus 100, as is described in FIG. 2. In another embodiment, the gas may be received from any suitable source. For example, the gas may be received from the insufflator 104, as is described in FIG. 2. As another example, the gas may be received from any other source that may provide a gas, such as, for example, a gas cartridge, a gas pump, a tank with a flow regulator, a centralized gas supply system in a hospital, or any other suitable gas source.

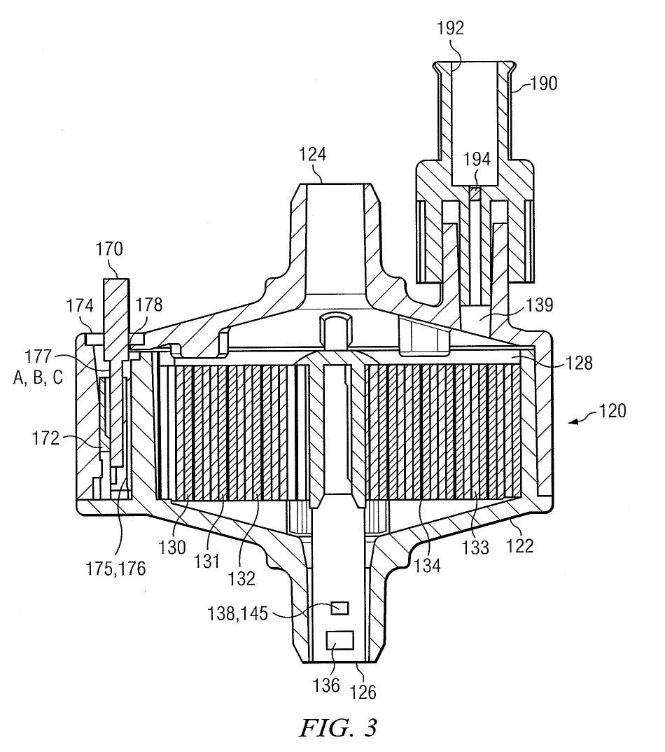

At step 18, the gas received from the source may be humidified and/or warmed. In one embodiment, the gas may be humidified and warmed by passing the gas through a chamber comprising a volume of a liquid. For example, the gas may be passed through the heater/humidifier 120, as is described in FIG. 3. As another example, the gas may be passed through any other suitable heater/humidifier. In another embodiment, the gas may be warmed to any particular temperature. For example, the temperature of the gas may be warmed so that it is within a temperature range as it exits the heating/humidifying chamber for delivery into the first incision of the patient. In one embodiment, the temperature range that the gas is warmed to is approximately 35°-40° C. For example, the gas may be warmed using a predetermined temperature set point, such as, 37° C. Other set points could be used without departing from the scope of the invention. Warming to a set point may result in a temperature range at the exit of the chamber. In another embodiment, the temperature that the gas is warmed to may be below 35° C. In a further embodiment, the temperature that the gas is warmed to may be above 40° C. In particular embodiments, the temperature range that the gas is warmed to may be approximately 28°-33° C., 30°-35° C., 32°-37° C., 37°-42° C., 39°-44° C., or any other suitable temperature range. In particular embodiments, the gas does not always need be warmed to a particular temperature range. For example, changes in the flow conditions or other influences may cause the temperature of the gas to be outside of the temperature range for a period of time. In some embodiments, the temperature may be adjustable and in others it may not.

The gas may be humidified to any particular relative humidity. For example, the gas may be humidified so that it is within a range of relative humidity at the exit of the heater/humidifier for delivery into the first incision of the patient. It may also be within any of the following humidity ranges as the gas enters the patient through the exit of a delivery device. The relative humidity level may be above 40%, above 50%, above 60%, above 70%, above 75%, above 80%, above 85%, or above 90% relative humidity. In further embodiments, the range of relative humidity may be between 65-80%, between 70-85%, between 75-90%, between 80-95%, or any other suitable range. In particular embodiments, the relative humidity of the gas does not always need to be within a particular range of relative humidity. For example, changes in the flow conditions or other influences may cause the relative humidity of the gas to be outside of the range of relative humidity for a period of time. In some embodiments, the relative humidity may be between 95% and 100%.

According to particular embodiments, the method may further include monitoring the relative humidity of the humidified gas. For example, in one embodiment, if the relative humidity of the gas falls below a predetermined relative humidity threshold, a signal may alert a user about the drop in relative humidity. In one embodiment, the method may further include injecting an additional amount of the liquid into the chamber. For example, the liquid may be injected into the chamber 128, as is discussed in FIG. 3. As such, in one embodiment, the additional amount of the liquid may increase the relative humidity of the gas.

In one embodiment, the liquid used to humidify the gas may include any suitable liquid. For example, the liquid may include water, such as sterile water. As another example, the liquid may include saline. In a further embodiment the liquid may include an anesthetic, antibiotic, or both. For example, the liquid may include lidocaine. In a further embodiment, the liquid may include an anticoagulant in order to prevent blood clots or clotting. For example, the liquid may include heparin or Angiomax. The liquid may also include a combination of water (or saline) and other substances, such as anesthetics, antibiotics, or anticoagulants. In a further embodiment, the liquid may include a gel substance containing water and other substances. In particular embodiments, the volume of the liquid may be contained in an absorbent material, such as is discussed in FIG. 3. In a further embodiment, the volume of the liquid may be maintained between two or more membranes, such as is also discussed in FIG. 3.

The method may further include filtering the gas received from the source. The filtering of the gas may be performed by any suitable filter. For example, the filtering of the gas may be accomplished using the filter 110 of FIG. 2. The gas may be filtered prior to the gas being humidified and warmed. The gas may be filtered after being humidified and warmed. In an additional embodiment, the gas may be filtered at any time before the humidified and warmed gas is delivered into the first incision.

In the illustrated embodiment, gas is both humidified and warmed. However, in some embodiments, the gas may only be humidified, while in others it may only be warmed. Warming may use any type of heater, while humidification may involve a volume of water in the chamber where the gas flows without use of a heater.

At step 20, the humidified and warmed gas is delivered into the first incision. The humidified and warmed gas may be delivered into the first incision using any suitable device. The humidified and warmed gas may be delivered into the first incision using any suitable surgical instrument attached to the apparatus 100 of FIG. 2. In such an example, the humidified and warmed gas may be delivered into the first incision using an endoscopic vein harvesting system, such as a VIRTUOSAPH endoscopic vein harvesting system or a MAQUET vasoview 7 xS endoscopic vessel harvesting system. Other types of vein harvesting systems may be used without departing from the scope of the invention. The humidified and warmed gas may travel through and/or over the surgical instrument inserted into the first incision of the patient. Accordingly, the humidified and warmed gas may come in contact with a blood vessel, such as the saphenous vein or the radial artery, of the patient.

In particular embodiments, by delivering the humidified and warmed gas into the first incision, the gas may create a space, such as a pocket, around the blood vessel. In such an embodiment, the space around the blood vessel may allow the blood vessel to be more properly viewed using a device, such as an endoscope, and may also provide room for separating one or more blood vessel connectors from the blood vessel.

At step 22, one or more blood vessel connectors are separated from a blood vessel. In one embodiment, the one or more blood vessel connectors may be separated from any suitable blood vessel, such as a saphenous vein or a radial artery. The blood vessel connectors may include any tissue, blood vessel branches, or any other biological components that are connected to the blood vessel.

The one or more blood vessel connectors may be separated from the blood vessel using at least one surgical instrument. For example, the at least one surgical instrument may include an endoscopic vein harvesting system, such as a VIRTUOSAPH endoscopic vein harvesting system or a MAQUET vasoview 7 xS endoscopic vessel harvesting system. Other types of vein harvesting systems may be used without departing from the scope of the invention. The at least one surgical instrument may include more than one surgical instrument, such as two or more surgical instruments. In a further embodiment, the at least one surgical instrument may include a single surgical instrument with various attachments. For example, the at least one surgical instrument may include an endoscopic vein harvesting system with a scissors attachment, and also with an extended length endoscope and dissection tip attachment. As such, the surgical instrument may be removed from the patient in order to put a different attachment onto the surgical instrument in order to perform a different aspect of the separation. Other types of vein harvesting systems, and other types of attachments may be used without departing from the scope of the invention. According to one embodiment, the at least one surgical instrument may include a surgical instrument attached to the apparatus 100 of FIG. 2.

The one or more blood vessel connectors may be separated from the blood vessel by separating one or more blood vessel connectors from the anterior area of the blood vessel and also separating one or more blood vessel connectors from the posterior area of the blood vessel. For example, the at least one surgical instrument may be used to first separate one or more blood vessel connectors from the anterior area of the blood vessel, and then may be used to separate one or more blood vessel connectors from the anterior area of the blood vessel, or vice versa. In one embodiment, all of the blood vessel connectors may be separated from the blood vessel. In a further embodiment, all of the blood vessel connectors may be separated from only a segment of the blood vessel. In such an embodiment, the blood vessel connectors may be separated from only the segment of the blood vessel that may be removed from the patient.

The one or more blood vessel connectors may be separated from the blood vessel using the at least one surgical instrument inserted through a second incision in the limb of the patient. The second incision may be (and typically is) the same incision as the first incision. The second incision may be a different incision than the first incision. The second incision in the limb of the patient may include an incision made in any area of the patient in order to access a portion of the limb of a patient. For example, the second incision in the limb of the patient may include an incision made in the upper body of the patient in order to access a blood vessel in the arm of the patient. As another example, the second incision in the limb of the patient may include an incision made in the lower body of the patient in order to access a blood vessel in the leg of the patient.

At step 24, a blood vessel segment is removed from the patient. The blood vessel segment may be removed from the patient using at least one surgical instrument. For example, the blood vessel segment may be removed from the patient using the same surgical instrument that is used to separate the one or more blood vessel connectors from the blood vessel. In a further example, the surgical instrument may be a different surgical instrument, or the same surgical instrument with a different attachment than that used to separate the one or more blood vessel connectors from the blood vessel. The blood vessel segment may be removed from the patient through a third incision in the limb of the patient. The third incision may be (and typically is) the same incision as the first incision and the second incision. The third incision may be a different incision than the first incision and/or the second incision. The third incision in the limb of the patient may include an incision made in any area of the patient in order to access a portion of the limb of the patient. For example, the third incision in the limb of the patient may include an incision made in the upper body of the patient in order to access a blood vessel in the arm of the patient. As another example, the third incision in the limb of the patient may include an incision made in the lower body of the patient in order to access a blood vessel in the leg of the patient.

The blood vessel segment may be removed from the patient using any suitable method. For example, the blood vessel segment may be removed from the patient using a “stab and grab” method. In such an example, a fourth incision may be made in the patient, allowing the blood vessel segment to be separated from the blood vessel. In such an example, the remaining section of the blood vessel may be tied off and the blood vessel segment may be removed from the patient through the first incision, the second incision, the third incision, or the fourth incision.

Removing the blood vessel segment from the patient may include multiple steps. For example, a first segment of the blood vessel may be removed, and then a second and/or any other suitable number of segments of the blood vessel may be removed. In one embodiment, removing the blood vessel segment from the patient may further include removing the blood vessel segment from different locations in the patient. For example, removing the blood vessel segment from the patient may include first removing a section of the saphenous vein located between the knee and the groin of the patient, and then further removing a section of the saphenous vein located between the knee and the ankle of the patient.

The blood vessel segment may include any length of the blood vessel. For example, the blood vessel segment may include the entire blood vessel, or a section of the blood vessel. For instance, the blood vessel segment may include the section of the saphenous vein that is located between the knee of the patient and the groin of the patient.

At step 26, a portion of the blood vessel segment is attached to a coronary blood vessel. The portion of the blood vessel segment may include the entire blood vessel segment, or any section of the blood vessel segment. For example, the blood vessel segment may be dissected into one or more sections. In one embodiment, the portion of the blood vessel segment may be attached to a coronary artery of the patient. For example, a first end of the portion of the blood vessel segment may be attached to a location on (or in) the coronary artery that is below (or above) a blockage in the coronary artery, and the second end of the portion of the blood vessel segment may be attached to a location on (or in) the coronary artery that is above (or below) the blockage in the coronary artery. In such an example, the portion of the blood vessel segment may allow blood to bypass the blockage in the coronary artery. Although the above example describes a coronary artery bypass being performed in order to bypass a blockage (e.g., occlusion) in the coronary artery, the coronary artery bypass may be performed in order to bypass any other suitable problem in the coronary artery, such as stenosis (e.g., narrowing), severe narrowing, partial narrowing, or partial blockage. In one embodiment, the portion of the blood vessel segment may be attached to a coronary blood vessel by grafting the portion of the blood vessel segment to the coronary blood vessel.

In particular embodiments, the portion of the blood vessel segment may be attached to the coronary blood vessel during a surgical operation that is separate from the surgical operation where the blood vessel segment was removed from the patient. In a further embodiment, the portion of the blood vessel segment may be attached to the coronary blood vessel in the same surgical operation where the blood vessel segment was removed from the patient. In a further embodiment, the portion of the blood vessel segment may be attached to a coronary blood vessel of a different patient than the patient from which the blood vessel segment was removed. For example, the blood vessel segment may be removed from a donor patient, and attached to a coronary blood vessel in a donee patient.

At step 28, the surgical method 10 ends. As is discussed above, in one embodiment, the surgical method 10 may allow a blood vessel segment to be in a better condition for use in a coronary blood vessel bypass. For example, the humidified and warmed gas may reduce desiccation of the outside of the blood vessel segment, causing it to be more moist and pliable.

In particular embodiments, although the surgical method 10 illustrates a surgical method for performing a coronary blood vessel bypass, the surgical method 10 may be performed for any other suitable blood vessel bypass. For example, the surgical method 10 may be used to conduct a femoral to below the knee arterial bypass surgery. In a further embodiment, the surgical method 10 may be used for any other surgical operation that may require a blood vessel segment removed from a patient. For example, the surgical method 10 may be used to conduct a liver blood vessel bypass or a kidney blood vessel bypass.

The steps illustrated in FIG. 1 may be combined, modified, or deleted where appropriate. Additional steps may also be added to the example operation. Furthermore, the described steps may be performed in any suitable order.

As is discussed above, FIG. 1 illustrates one embodiment of a surgical method for performing a coronary blood vessel bypass. FIGS. 2-7, on the other hand, illustrate particular embodiments of one or more apparatuses that may be used during the method of FIG. 1. Although FIGS. 2-7 illustrate particular embodiments of one or more apparatuses that may be used in the method of FIG. 1, any other suitable apparatuses may be used in the method.

Referring to FIG. 2, one embodiment of an apparatus for treating or conditioning insufflation gas is shown generally at reference numeral 100. The apparatus 100 is adapted to receive gas from a gas source (high or low pressure, high or low flow rate), such as insufflation gas from an insufflator 104 for delivery into a body of a patient. The apparatus comprises a filter 110, a heater/humidifier 120, and a control module 140. A tubing set is provided to connect the various components of the apparatus together. Specifically, a first tube segment 160 connects the outlet of the insufflator 104 to the inlet tubing of the filter 110 via a male Luer lock 166 or any appropriate adapter compatible with the insufflator outlet port. A second tube segment 162 connects the outlet of the filter 110 to the inlet of the heater/humidifier 120. A third tube segment 164 connects the outlet of the heater/humidifier 120 by a male Luer lock 168 (or other appropriate fitting adapter) to a gas delivery device (not shown), such as a trocar, verres needle, endoscope, any vein harvesting device, such as a VIRTUOSAPH endoscopic vein harvesting system or a MAQUET vasoview 7 xS endoscopic vessel harvesting system, or a tube that enters a body cavity or space that delivers the filtered, humidified and warmed gas into the body of a patient. The tubing of the tube segments 160, 162 and 164 may be flexible and sufficiently long to permit the insufflator 104 and control module 140 to be placed at a convenient distance from a patient undergoing laparoscopic or other surgery or procedure requiring gas distention, while the heater/humidifier 120 may be placed within 12 inches of the patient.

The filter 110 is optional, but may be a particulates filter (for example a BF201 filter from AG Industries, with a HA-8141 filter media from Hollingsworth & Vose) having a pore size preferably small enough to exclude all solid particles and bacterial or fungal agents that may have been generated in a gas supply cylinder, such as a carbon dioxide cartridge, or the insufflator 104 (e.g., 0.5 micron or less, for example, about 0.3 micron). As another example, the filter 110 may be a DDF5500M02C-LM particulates filter from Porous Media. In one embodiment, the filter 110 is a hydrophobic filter. In another embodiment, the filter 110 is a hydrophilic filter. In a further embodiment, decreasing the pore size of filter 110 below 0.3 micron may cause a concomitant increase in pressure drop of gas, and thus flow rate may be reduced significantly. In one embodiment, the filter 110 may be disposed in the apparatus 100 in any suitable location. For example, the filter 110 may be disposed in the apparatus 100 in a location where the gas passes through the filter 110 before entering the heater/humidifier 120. As such, the gas may be filtered prior to being humidified and warmed. In another embodiment, the filter 110 may be disposed in the apparatus 100 in a location where the gas enters the filter 110 after exiting the heater/humidifier 120. As such, the gas may be filtered after being humidified and warmed.

In one embodiment, the heater/humidifier 120 is connected by tubing to a gas delivery device, such as a surgical instrument, so that the gas travels a short distance from the outlet of the heater/humidifier 120 to the conduit or connection to the interior of a patient. The purpose of this arrangement may be to allow gas to be delivered to the patient while still at a temperature and water content sufficiently close to the physiological interior body temperature and humidity. That is, the apparatus according to the disclosure may reduce thermodynamic cooling of medical gases in transit to the patient, because it provides a highly efficient humidifying and warming chamber that, as a result of its efficiency, can be quite compact and thus be positioned very near to the patient.

In one embodiment, the control module 140 is contained within an electrical housing 210 and is connected to the heater/humidifier 120 by several wire pairs contained within an insulated electrical cable 170. In particular, the cable 170 has a connector 172 at one end that electrically connects into a receptacle of the housing 210 for the control module 140, and at the other end it is electrically connected to the heater/humidifier 120 by a sealed electrical feedthrough 174. In one embodiment, the cable 170 is attached to the tube segment 162 by a plastic tape or clip 176. In another embodiment, the cable 170 is attached to the tube segment 162 by heat seal, extrusion, ultrasonic welding, laser transmission welding, glue or is passed through the interior of tube segment 162.

The control module 140 and associated components in the heater/humidifier 120 may be powered by an AC-DC converter 180. In one embodiment, the AC-DC converter 180 has an output that is connected by a plug connector 182 into a receptacle of the housing 210 to the control module 140, and has a standard AC wall outlet plug 184 that can be plugged into standard AC power outlets. For example, the AC-DC converter 180 is plugged into an AC power strip that is provided on other equipment in an operating room. In another embodiment, electrical power for the apparatus is provided by a battery or photovoltaic source. In further embodiments, circuitry may be provided in the control module 140 that operates on AC signals, as opposed to DC signals, in which case the control module 140 could be powered directly by an AC outlet.

In one embodiment, the heater/humidifier 120 has a charging port 190 that is capable of receiving a supply of liquid therethrough to charge the humidification means (described hereinafter) with liquid. For example, a syringe 200 containing a predetermined volume of liquid is introduced into the charging port 190 to inject liquid into the heater/humidifier 120 for an initial charge or re-charge of liquid. The apparatus 100 may be sold with the heater/humidifier 120 pre-charged with a supply of liquid such that an initial charge is not required for operation.

Modifications, additions, or omissions may be made to the apparatus 100 without departing from the scope of the invention. The components of the apparatus 100 may be integrated or separated. Moreover, the operations of the apparatus 100 may be performed by more, fewer, or other components. For example, the operations of the heater/humidifier 120 may be performed by more than one component. As used in this document, “each” refers to each member of a set or each member of a subset of a set.

Turning to FIG. 3, one embodiment of the heater/humidifier 120 will be described in greater detail. In this embodiment, the heater/humidifier 120 comprises a housing 122 having an (entry port) inlet 124 and an (exit port) outlet 126. The housing 122 defines a chamber 128 that contains elements for substantially simultaneously humidifying (hydrating) and warming the gas supplied through the inlet 124, as well as means for sensing the temperature of the gas and the relative humidity of the gas as it exits the chamber 128.

In this embodiment, within the chamber 128, there is provided a humidification means that may be comprised of one or more layers of liquid-retaining or absorbing padding or sponge material, shown at reference numerals 130, 131 and 132. It should be understood that the number, spacing and absorbency of the liquid-retaining layers 130, 131 and 132 may be varied according to specific applications. Three liquid-retaining layers are shown as an example. The material of the liquid-retaining layers 130, 131 and 132 can be any desirable liquid-retaining material, such as a hydrophyilic material (e.g., Knowlton 1430 by Hydration media). The pore size of the selected material may be chosen according to a balance of water retention capabilities and low pressure drop considerations. The larger the pore size, the greater the water retention capability for humidification.

In particular embodiments, the humidification means may consist of a chamber of liquid (without liquid-retaining layers) having one or more semi-permeable membranes on opposite ends to allow gas to pass therethrough. The liquid in the chamber could be warmed by a heating jacket placed around the chamber to thereby humidify and warm the gas passed therethrough. In another embodiment, the humidification means may be disposed in any suitable location and in any suitable manner in the chamber. For example, the humidification means may be disposed in the chamber so that gas flows through the humidification means and/or flows over, under, and/or around the humidification means. In a further embodiment, the chamber may include a further humidification means that is separate from the humidification means. For example, the chamber may further include a humidification chamber that may aerosolize a liquid, such as an anesthetic discussed above in FIG. 1, into the gas. In such an example, the humidification chamber may include any suitable amount of the liquid, such as 2 cc. In a further embodiment, the humidification means may include any suitable amount of the liquid, such as 0.1 cc-15 cc. In one embodiment, the amount of the liquid included in the humidification means may be determined based on the dimensions of the humidification means and/or the holding capacity of any material in the humidification means.

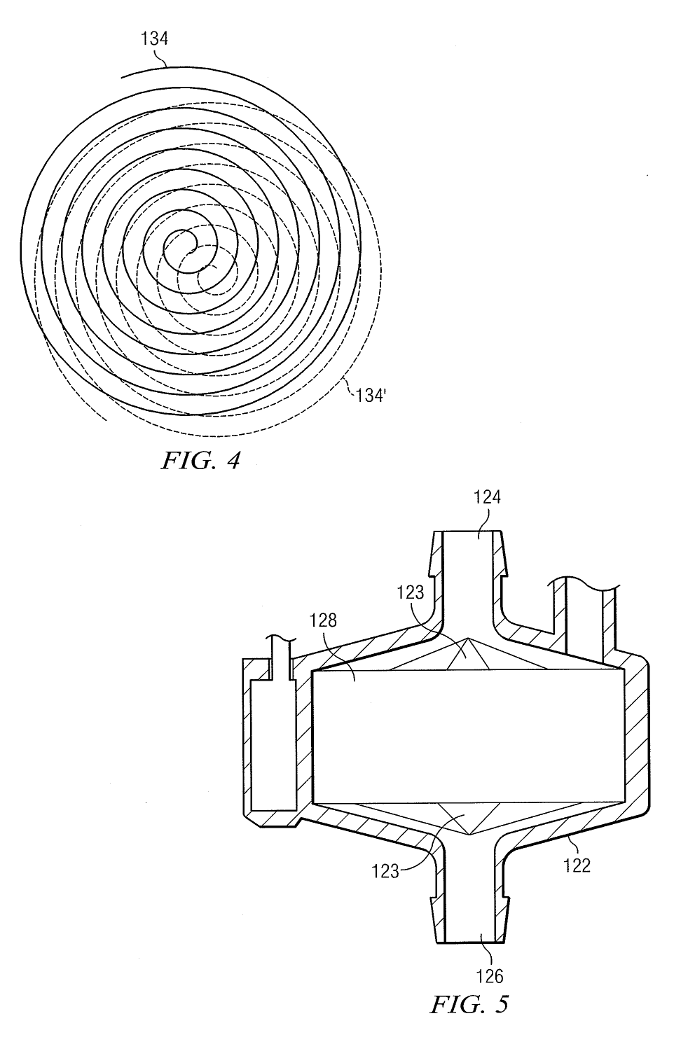

In one embodiment, the heating means in the heater/humidifier 120 comprises at least one heating element 134 positioned in the housing (substantially) co-located with the humidification means, such as between the liquid-retaining layers 130 and 131. The heating element 134 may be an electrically resistive wire, for example, and is described in more detail hereinafter in conjunction with FIG. 4. The heating element 134 may be positioned within the humidification means insofar as it is placed preferably between liquid-retaining layers. The heating element 134 warms the insufflation gas supplied through the inlet, under control of a heat control signal supplied by the control module 140, substantially simultaneous with the humidification of the gas as the gas passes through the chamber 128. Additional heating elements may be disposed within the chamber.

Other types of heating elements may be used without departing from the scope of the invention. The heating elements may be placed anywhere in the chamber. In some embodiments, multiple chambers may be used to serially humidify and warm the gas, or serial chambers could both humidify and warm or apply a pharmacologic agent.

According to one embodiment, in order to sense the temperature and humidity of the gas as it exits the heater/humidifier 120, a temperature sensor 136 and a relative humidity sensor 138 may be provided. In one embodiment, the temperature sensor 136 may be provided anywhere within the flow of gas. In a further embodiment, the temperature sensor 136 may be provided in any other location that allows it to sense the temperature of the gas. In one embodiment, the temperature sensor 136 is a thermistor. In one embodiment, the temperature sensor 136 may be accurate to within about 0.2° C. In particular embodiments, the temperature of the gas may be sensed after the gas has been humidified so that any change in the temperature of the gas as it is humidified is corrected at that point in the apparatus, thereby compensating for enthalpy changes. In an alternative embodiment, temperature of the gas can be sensed indirectly by sensing the temperature of the heater. Infrared sensors could also be used.

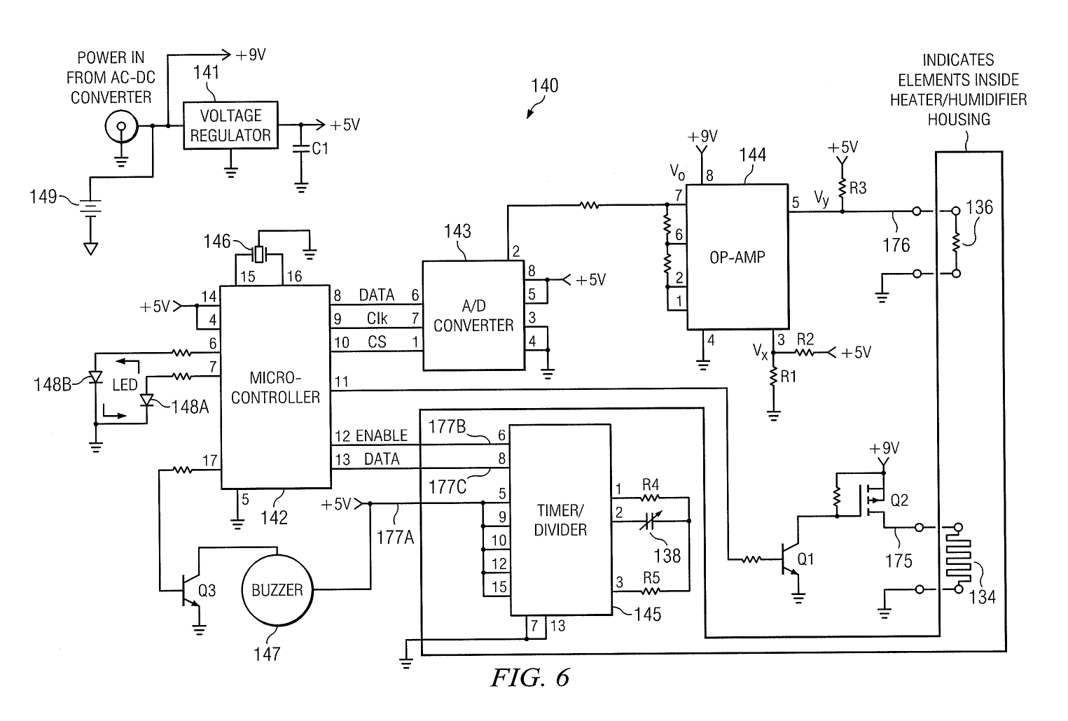

In one embodiment, the humidity sensor 138 is positioned in the flow path of gas exiting the chamber 128, preferably downstream from the heating element 134 either between liquid-retaining layers or on the downstream side of the liquid-retaining layers, proximate the exit port 126 of the housing 122. Humidity sensor 138 is optional as is a temperature sensor. The humidity sensor 138 is preferably not in contact with a liquid-retaining layer. FIG. 3 shows the humidity sensor 138 distal to the liquid-retaining layers, separated from the liquid-retaining layer 132 by a porous mesh (plastic or metal) layer 133 that extends across the interior of the housing 122. The humidity sensor 138 actually is generally not in contact with the porous mesh layer 133, but is spaced therefrom as well. The humidity sensor 138 may be a humidity-sensitive capacitor sensor, such as a capacitive humidity sensor manufactured by Philips Corporation, which changes capacitance in response to humidity changes. Other humidity sensors can also be used. The humidity sensor 138 measures the relative humidity of the gas as it passes through the chamber 128 to enable monitoring of the gas humidity, and in order to provide an indication of the amount of liquid remaining in the humidification means, e.g., in liquid-retainer layers 130, 131 and 132. As will be explained hereinafter, a timer/divider integrated circuit (IC) 145 (FIG. 6), is connected to the humidity sensor 138 and may be disposed within the housing 122 together and substantially co-located with the humidity sensor 138.

According to one embodiment, electrical connections to the components inside the housing 122 of the heater/humidifier 120 are as follows. A ground or reference lead (not specifically shown) is provided that is connected to each of the temperature sensor 136, heating element 134 and humidity sensor 138-timer/divider 145. A wire 175 (for a positive lead) electrically connects to the heating element 134 and a wire 176 (for a positive lead) electrically connects to the temperature sensor 136. In addition, three wires 177A, 177B and 177C (shown in more detail in FIG. 6) electrically connect to the humidity sensor 138-timer divider circuitry, wherein wire 177A carries a DC voltage to the timer/divider 145, wire 177B carries an enable signal to the timer/divider 145, and wire 177C carries an output signal (data) from the timer/divider 145. All of the wires are fed from the insulated cable 170 into the feedthrough 174 and through small holes in the housing 122 into the chamber 128. The feedthrough 174 is sealed at the opening 178 around the cable 170. The optional charging port 190 is attached to a lateral extension 139 of the housing 122. The charging port 190 comprises a cylindrical body 192 containing a resealable member 194. The resealable member 194 permits a device to be inserted therethrough, but seals around the exterior of the device. This allows a volume of liquid (sterile water, saline, etc.) to be delivered into the chamber 128 without releasing the liquid already contained therein. The resealable member 194 is, for example, a Luer lock check valve, such as P/N B900-SSM41 manufactured by NP Medical or P/N SCV23050 manufactured by Value Plastics. Alternatively, the charging port may be embodied by a one-way valve, a sealable port, a screw cap, a cap with a slit to permit the introduction of a syringe or other device, such as a SAFELINE injection site, part number NF9100, manufactured by B. Braun Medical Inc., or any other covering material or member capable of permitting the introduction of a device and preventing the backflow of contained liquid or gas. In one embodiment, the chamber 128 may contain approximately 3 to 8 cubic centimeters (cc) (but possibly as much as 10 cc) of liquid, and it may be desirable that the gas have a dwell time within the chamber of at least approximately 0.01 to 1.0 sec. A liquid volume of 8 cc in the chamber 128 will usually be adequate for conditioning approximately 180 liters of insufflation gas at a relative humidity of 80-95%. The control module 140, however, may issue a warning when the humidity of the gas being treated by the heater/humidifier 120 drops below a predetermined relative humidity, as explained hereinafter. Charging ports may be included for recharging or charging of a pharmacologic agent (such as an anesthetic or antibiotic).

In one embodiment, the housing 122 may have a length to width ratio of about 1:2 to about 1:10. In a further embodiment, the housing 122 may have a length to width ratio of about 1:3 to about 1:4. In one embodiment, the length of the housing 122 may be from about 0.5 cm to about 1.5 cm, and the diameter may be about 3.0 cm to about 5.0 cm. For example, one embodiment of the housing 122 is approximately 3.5 centimeters (cm) in diameter and 1.0 cm thick. The length and width of chamber 128 can be varied such that proper humidification and warming occur. In one embodiment, an elongated housing configuration would permit the heater/humidifier 120 to be less intrusive to the medical attendant or surgeon and also be freely movable with respect to other equipment in or around the apparatus 100. Any length to width ratio may be used without departing from the scope of the invention.

In one embodiment, the desirable width and diameter of the chamber may also be dependent upon the rate of gas flow from insufflator 104, which is usually from about 1-20 liters/minute, and upon the pressure desired to be maintained, which is affected more by the diameter of chamber 128 than by its length. A person of ordinary skill in the art, given the teachings and examples herein, can readily determine suitable dimensions for chamber 128 without undue experimentation.

Modifications, additions, or omissions may be made to the heater/humidifier 120 without departing from the scope of the invention. The components of the heater/humidifier 120 may be integrated or separated. Moreover, the operations of the heater/humidifier 120 may be performed by more, fewer, or other components. For example, the operations of the heating element 134 may be performed by more than one component.

Referring to FIG. 4, one embodiment of the heating element 134 is shown in more detail. The heating clement 134 is an electrically resistive wire that is disposed in the housing 128 in a concentrical coil configuration having a predetermined number of turns, such as 6-8 turns. In another embodiment, a second heating element 134′ is provided that is arranged with respect to the heating element 134 such that its coils are offset from those of the first heating element, relative to the direction of gas flow through the chamber. In one embodiment, if two or more heating elements are employed, they are preferably spaced from each other in the chamber of the heater/humidifier by approximately 3-4 mm. The first and second heating elements 134 and 134′ can be coiled in opposite directions relative to each other. This arrangement allows for maximum contact of the gas flowing through the chamber with a heating element. Other non-coiled configurations of the heating element 134 are also suitable.

Modifications, additions, or omissions may be made to the heating element 134 without departing from the scope of the invention. The components of the heating element 134 may be integrated or separated. Moreover, the operations of the heating element 134 may be performed by more, fewer, or other components.

Turning to FIG. 5, another feature of one embodiment of the heater/humidifier 120 is illustrated. At the inlet and/or outlet of the housing 122, fluted surfaces 123 may be provided to facilitate complete dispersion of gas as it is supplied to the heater/humidifier 120. This improves the fluid dynamics of the gas flow through the chamber 128 to ensure that the gas is uniformly humidified and warmed as it flows through the chamber 128.

Referring to FIG. 6, one embodiment of the control module 140 will be described in detail. The control module 140 contains an example of monitoring circuitry and an example of control circuitry for the apparatus 100, and comprises a voltage regulator 141, a microcontroller 142, an A/D converter 143, a dual operational amplifier (hereinafter “op-amp”) module 144, and a timer/divider 145. The monitoring circuit portion of the control module 140 comprises the combination of the microcontroller 142 and timer/divider 145. The control circuit portion of the control module 140 consists of the microcontroller 142, A/D converter 143 and op-amp module 144. The monitoring circuit monitors the relative humidity of gas exiting the chamber based on a signal generated by the timer/divider 145. The control circuit monitors the temperature of the gas exiting the chamber and in response, controls electrical power to the heating element to regulate the temperature of the gas to a temperature within a range of temperatures. In one embodiment, while the temperature of the gas exiting the chamber is actively controlled, the relative humidity of the gas in the chamber may not be actively controlled. For example, in one embodiment, the relative humidity of the gas may be monitored and an alert may be generated when it drops below a predetermined threshold so that appropriate action can be taken, such as replenishing the heater/humidifier with liquid.

FIG. 6 shows that, in one embodiment, several components may be located within the electrical housing 210 (FIG. 2), whereas other components may be located within the housing of the heater/humidifier 120 (FIG. 3). In particular, the timer/divider 145 and the associated resistors R4 and R5 may be located inside the housing 122 of the heater/humidifier 120, together with the humidity sensor 138 in a circuit package that includes the humidity sensor 138 exposed on one or more surfaces thereof More specifically, the timer/divider 145 is co-located with humidity sensor 138. This configuration minimizes timing error by stray wiring inductance and capacitance (sensor kept close to active circuits of timer/divider 145). In addition, by co-locating the timer/divider 145 and humidity sensor 138, the need for interconnecting wires is eliminated, thereby avoiding undesirable signal radiation. However, any location arrangement of components is allowable.

The voltage regulator 141 receives as input the DC output of the AC-DC converter 180 (FIG. 2), such as for example, 9 V DC, that is suitable for use by the analog components of the control module. The voltage regulator 141 regulates this voltage to generate a lower voltage, such as 5 V DC, for use by the digital components of the control module. The capacitor C1 at the output of the voltage regulator 141 serves to filter out any AC components, as is well known in the art. Alternatively, a suitable DC voltage is provided by a battery or photovoltaic source shown at reference numeral 149.

In one embodiment, the microcontroller 142 is a PIC16C84 integrated circuit microcontroller that controls system operation. A ceramic resonator 146 (4 MHz) is provided to supply a raw clock signal to pins 15 and 16 of the microcontroller 142, which uses it to generate a clock signal for the signal processing functions explained hereinafter.

The op-amp 144 module may be coupled (by wire 176) to the temperature sensor 136 (thermistor). The op-amp module 144 is, for example, a LTC1013 dual low-input-offset-voltage operational amplifier integrated circuit that includes two op-amps, referred to hereinafter as op-amp A and op-amp B. The non-inverting input of the op-amp A of the op amp module 144 is pin 3, and pin 2 is the inverting input. The output of op-amp A is pin 1. Op-amp A of the op-amp module 144 is used to buffer the output voltage of the voltage divider formed by resistors R1 and R2. The buffered output voltage, referred to as Vx in FIG. 6, is applied to op-amp B in the op-amp module 144. Op-amp B is configured as a non-inverting-with-offset amplifier with a gain of 21.5, and also receives as input the output of the temperature sensor 136, adjusted by resistor R3, shown as voltage Vy in the diagram. The output voltage of op-amp B is at pin 7, referred to as Vo in FIG. 6. The output voltage Vo is equal to 21.5 Vy−20.5 Vx, which is inversely proportional to the gas temperature in the housing of the heater/humidifier. The output voltage Vo ranges between 0-5 V DC, depending on the temperature of the gas in the chamber.

In one embodiment, the A/D converter 143 is an ADC 0831 integrated circuit analog-to-digital converter that receives as input at pin 2, the output Vo of the op-amp module 144. The A/D converter 143 generates a multi-bit digital word, consisting of 8 bits for example, that represents the output voltage Vo, and is supplied as output at pin 6, which in turn is coupled to I/O pin 8 of the microcontroller 142. The microcontroller 142 commands the A/D converter 143 to output the digital word by issuing a control signal on I/O pin 10 which is coupled to the chip select pin 1 of the A/D converter 143. Moreover, the microcontroller 142 controls the rate at which the A/D converter 143 outputs the digital word by supplying a sequence of pulses on pin 9 applied to clock input pin 7 of the A/D converter 143. The “unbalanced bridge” values of resistors R1, R2 and R3 may be chosen to produce a 0-5 V DC output over gas temperatures from approximately 20° C. to approximately 45° C. Since the bridge and the reference for the A/D converter 143 are provided by the same 5 V DC source, error due to any reference voltage shift may be eliminated.

The timer/divider 145 is, for example, a MC14541 precision timer/divider integrated circuit. The humidity sensor 138 is connected to pin 2 and to resistors R4 and R5 as shown. In response to an enable signal output by the microcontroller 142 on pin 12 that is coupled to timer/divider pin 6, the timer/divider 145 generates an output signal that oscillates at a rate determined by the value of the resistor R4, the capacitance of the humidity sensor 138 (which varies according to the relative humidity of the gas inside the heater/humidifier housing) and a predetermined divider constant. For example, the divider constant is 256. Specifically, the output signal of the timer/divider 145 is a square wave oscillating between 0 V (“low”) and 5 V (“high”) at a frequency of approximately 1/[256*2.3*R4ₜ*Cₜ]Hz, where R4ₜ is, for example, 56 kOhms, and Cₜ is the capacitance at some time (t) of the relative humidity sensor 138 depending on the relative humidity of the gas in the chamber. For example, the humidity sensor manufactured by Phillips Electronics, referred to above, can measure between 10-90% RH (relative humidity), where Cₜ at 43% RH is 122 pF (+—15%), with a sensitivity of 0.4+/−0.5 pF per 1% RH. The output signal of the timer/divider 145 appears at pin 8, which is coupled to the I/O pin 13 of the microcontroller 142. Thus, the timer/divider 145 may essentially be an oscillator circuit connected to the humidity sensor that generates an output signal with a frequency dependent on a capacitance of the humidity sensor. In one embodiment, any oscillator circuit that can generate as output a signal whose frequency is dependent on a variable capacitance may be suitable for the timer/divider 145.

In one embodiment, the microcontroller 142 computes a measure of the relative humidity of the gas inside the heater/humidifier housing by timing or measuring a characteristic of the output signal of the timer/divider 145. For example, the microcontroller 142 measures the time duration of one of the phases of the output signal of the timer/divider 142, such as the “high” phase which is approximately 1/2*[256*2.3*R4ₜ*Cₜ]. This time duration may be indicative of the relative humidity of the gas in the chamber of the heater/humidifier since the rate of the oscillation of the timer/divider depends on the capacitance of the humidity sensor 138, as explained above. For example, for a change in RH of 10-50% and/or 50 to 90%, there is a 13% change in the duration of the “high” phase of the timer/divider output signal. The microcontroller 142 monitors the relative humidity of the gas exiting the chamber in this manner and when it drops below a predetermined relative humidity threshold (indicated by a corresponding predetermined change in the oscillation rate of the timer/divider 145), the microcontroller 142 generates a signal on pin 17, called a recharge signal, that drives transistor Q3 to activate an audible alarm device, such as buzzer 147. The buzzer 147 generates an audible sound which indicates that the relative humidity of the gas in the heater/humidifier has dropped below the predetermined threshold and that it is necessary to recharge the heater/humidifier with liquid. In one embodiment, the predetermined relative humidity threshold corresponds to a minimum level for a desirable relative humidity range of the gas exiting the heater/humidifier, and may be 40%, for example. In a further example, the predetermined relative humidity threshold may be 20%, 25%, 30%, 35%, 50%, 60%, 70%, 75%, 80%, 85%, 90%, 95%, or any other suitable percentage of relative humidity. The predetermined relative humidity threshold may be an adjustable or programmable parameter in the microcontroller 142. Optionally, the microcontroller 142 may generate another warning signal at the output of pin 7 to illuminate an light emitting diode (LED) 148A, thereby providing a visual indication of the humidity dropping below the predetermined relative humidity threshold in the heater/humidifier, and the need to recharge the heater/humidifier 120 with liquid. In a further embodiment, the microcontroller 142 generates a trouble or warning signal output at pin 6 to drive LED 148B (of a different color than LED 148A, for example) when there is either a “code fault” in the microcontroller 142 (an extremely unlikely occurrence) or when the relative humidity of the gas in the heater/humidifier is less than a critical relative humidity threshold (lower than the predetermined relative humidity threshold), such as 10%, or any other relative humidity percentage that is lower than the predetermined relative humidity threshold. In either case, power to the heating element 134 may be terminated in response to the warning signal.

In one embodiment, the microcontroller 142 also controls the heating element 134 in order to regulate the temperature of the gas inside the heater/humidifier. Accordingly, the microcontroller 142 processes the digital word supplied by the A/D converter 143 to determine the temperature of the gas inside the heater/humidifier housing. In response, the microcontroller 142 generates a heat control signal on the output pin 11 that drives transistor Q1, which in turn drives the MOSFET power transistor Q2, that supplies current to the heating element 134. The temperature of the gas inside the heater/humidifier may be regulated by the microcontroller 142 so that it is within a temperature range as it exits the heater/humidifier for delivery into the body of the patient. In one embodiment, the temperature range that the gas is regulated to is approximately 35°-40° C. For example, the gas may be regulated to a predetermined temperature set point, such as, 37° C., resulting in a smaller temperature range. Other set points could be used without departing from the scope of the invention. Regulating to a set point may result in a temperature range at the exit of the chamber. In another embodiment, the temperature that the gas is regulated to may be below 35° C. In a further embodiment, the temperature that the gas is regulated to may be above 40° C. In particular embodiments, the temperature range that the gas is regulated to may be approximately 28°-33° C., 30°-35° C., 32°-37° C., 37°-42° C., 39°-44° C., or any other suitable temperature range. In some embodiments, the temperature may be adjustable and in others it may not. The temperature of the gas may not always be within the temperature range. Changes in flow conditions, start up, or other circumstances may lead to periods where the gas temperature is outside of a desirable range.

As mentioned above, when the relative humidity inside the heater/humidifier falls below a critical threshold as determined by the monitoring circuit portion of the control module 140, the control circuit portion in response terminates power to the heating clement 134 to prevent the delivery of warm gas that is extremely dry. The temperature of the gas may not always be within the temperature range. Changes in flow conditions, start up, or other circumstances may lead to periods where the gas temperature is outside of a desirable range.

In one embodiment, the circuitry for monitoring the relative humidity of the gas can be embodied by other circuitry well known in the art. In addition, while the control module 140 has been described as having a single microcontroller 142 for monitoring signals representing temperature and relative humidity of the gas exiting the chamber, and for controlling the heating element to control the temperature of the gas, it should be understood that two or more microcontrollers could be used. For example, one microcontroller may dedicated to each of the individual functions. In addition, the functions of the microcontroller 142 could be achieved by other circuits, such as an application specific integrated circuit (ASIC), digital logic circuits, a microprocessor, a digital signal processor, or any other suitable circuit.

In one embodiment, the volume of gas that can be conditioned with a full supply of liquid in the heater/humidifier may depend on the flow rate and pressure used during a procedure. In particular embodiments, the apparatus may be designed to accommodate different anticipated needs for particular procedures. As an example, the chamber of the heater/humidifier may be designed to hold 8 to 10 cc of liquid that can humidify 180 liters of gas at a relative humidity level of 80% or more. The microcontroller 142 is programmable to issue the recharge signal when the humidity of the gas drops below the predetermined relativity humidity threshold, independent of the flow rate or pressure of the insufflation gas supply. Preferably, the predetermined relativity humidity threshold is set so that brief periods of high pressure or high flow rate do not cause this threshold to be triggered, because the humidity level will return to greater-than-threshold levels shortly after the high pressure/flow rate periods ends.

Modifications, additions, or omissions may be made to the control module 140 without departing from the scope of the invention. The components of the control module 140 may be integrated or separated. Moreover, the operations of the control module 140 may be performed by more, fewer, or other components. For example, the operations of the microcontroller 142 may be performed by more than one component.

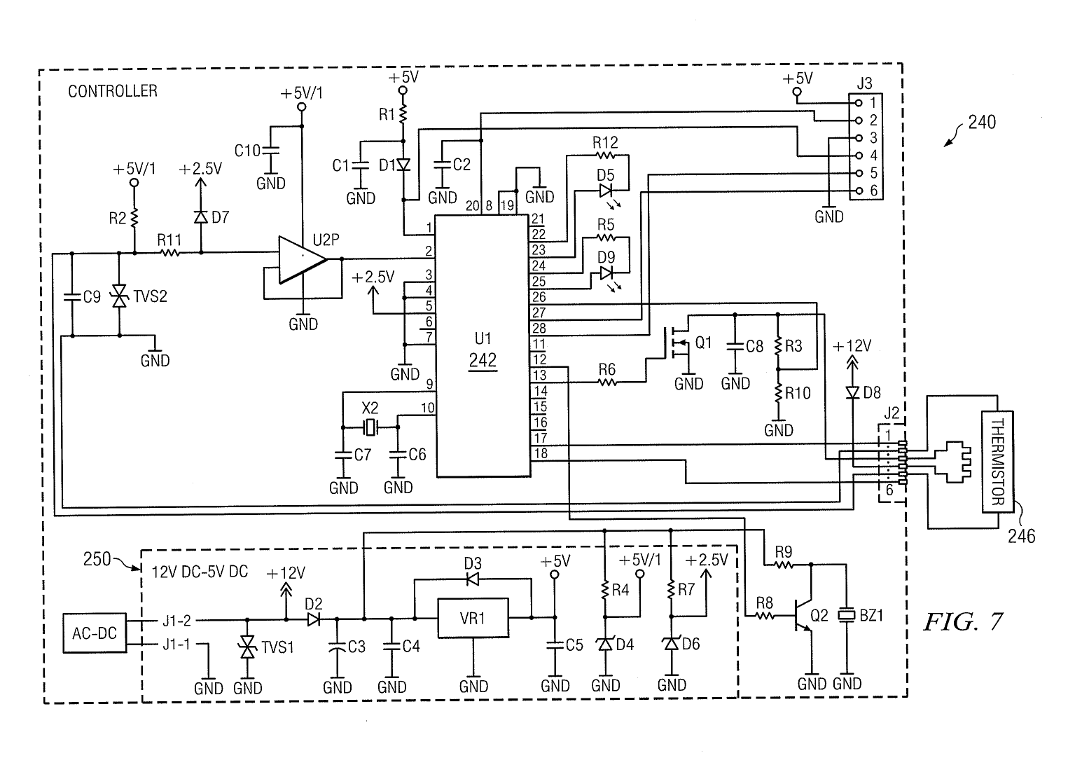

In a further embodiment, other various control modules may be used in the apparatus 100 instead of the control module 140 of FIG. 6. For example, FIG. 7 illustrates a control module 240 that may be used in the apparatus 100. In one embodiment, several components of the control module 240 may be located within the electrical housing 210 (FIG. 2), whereas other components may be located within the housing of the heater/humidifier 120 (FIG. 3). However, any location arrangement of the components is allowable.

In one embodiment, the microprocessor 242 is a PIC16F873SO microprocessor that controls the operation of the apparatus 100. The microprocessor 242 receives a 5 V DC from a voltage divider. A clock signal is generated by a 4 MHz resonator X2 tied to pins 9 and 10. An audible buzzer BZ1 whose frequency is determined by a transistor Q2 and two visible LED lights D5 and D9 are controlled by the microprocessor 242 to signal warnings or signal shutdown of the apparatus 100 due to fault conditions that the software scans for.

The microprocessor 242 also controls a heater (not shown), such as a resistive heater, in order to regulate the temperature of the gas inside the heater/humidifier. The heater is activated by pin 13 of the microprocessor 242 that triggers a transistor Q1 to allow a path to ground, completing a 12 V DC circuit. Temperature feedback is provided through pin 27 to the microprocessor 242. Regulation of the temperature by the microprocessor 242 may be software driven. In one embodiment, any suitable software may be used. Furthermore, additional programming of the control module 240 may occur at a programming unit J3. According to the illustrated embodiment, a proportional-integral-derivative (PID) temperature control loop may also be utilized in order to regulate the temperature.

In order to regulate the temperature, the microprocessor 242 receives imbedded temperature feedback from a temperature sensor (thermistor) 246. For example, a temperature sensing loop in the control module 240 may utilize a 5 V DC signal from a voltage divider. This signal is linked to the temperature sensor 246, and is fed to the positive input on an operational amplifier (op amp) U2P. One or more capacitors and one or more resistors may be used to stabilize the signal and reduce the chance of “noise.” The amplified signal is then fed to input 2 on the microprocessor 242.

The temperature sensor 246 may be placed in the gas stream. In one embodiment, the temperature sensor 246 may be placed in a location where it does not contact a humidification means of the apparatus 100. The temperature sensor 246 (and the heater) may be connected to the control module 240 via insulated wire and one or more connectors. In the illustrated embodiment, the temperature sensor 246 is connected to the control module 240 at J2 (pins 2 and 5), and the heater is connected to the control module 240 at J2 (pins 3 and 4).

The control module 240 may further include a 120 V AC to 12 V DC converter (power supply). This allows for the control module 240 to utilize standard 120 V AC wall power while using lower/safer voltage 12 V DC for warming. Power is further limited by a voltage regulating circuit 250. For example, the voltage regulating circuit 250 regulates the 12 V DC to a lower voltage, such as 5 V DC, for use by the temperature sensing circuit and the microprocessor 242.

Although FIG. 7 does not illustrate a humidity sensor, any suitable humidity sensor, such as the humidity sensor (and the related humidity sensing/monitoring components) discussed above in FIG. 6, may be used in conjunction with the control module 240 of FIG. 7. Furthermore, the circuitry for monitoring the relative humidity of the gas can be embodied by other circuitry well known in the art. In another embodiment, FIG. 7 may not include a humidity sensor and/or may not include circuitry for monitoring the relative humidity of the gas. Furthermore, while the control module 240 has been described as having a single microprocessor 242 for monitoring signals representing temperature and for controlling the heating element to regulate the temperature of the gas, it should be understood that two or more microprocessors could be used. For example, one microprocessor may dedicated to each of the individual functions. In addition, the functions of the microprocessor 242 could be achieved by other circuits.

Modifications, additions, or omissions may be made to the control module 240 without departing from the scope of the invention. The components of the control module 240 may be integrated or separated. Moreover, the operations of the control module 240 may be performed by more, fewer, or other components. For example, the operations of the microprocessor 242 may be performed by more than one component.

With reference to FIGS. 2 and 3, one embodiment of the setup and operation of the apparatus 100 will be described. The AC/DC converter 180 is plugged into a 110 V AC power source, such as a wall outlet or a power strip. The control module 140 is connected to the AC/DC converter 180. In another embodiment, the apparatus 100 may be powered by a battery or photovoltaic source. The heater/humidifying tubing set is then installed by attaching one end of the tube segment 160 to the outlet of the insufflator 104 by the Luer lock 166. The tube segments 160, 162 and 164 may be pre-attached to the filter 110 and the heater/humidifier 120 for commercial distribution of the apparatus 100. The cable 170 is installed into the electrical housing 210 of control module 140 by the connector 172. The heater/humidifier 120 is charged with a supply of liquid by the syringe 200. For example, 8 cc of a liquid, such as sterile water or saline, is drawn into the syringe 200. The syringe 200 is then inserted into the charging port 190 so that a needle or cannula of the syringe 200 penetrates the resealable member 194 (FIG. 3) and the liquid is injected into the heater/humidifier 120 to be absorbed by the liquid-retaining layers. The syringe 200 is then removed from the charging port 190, and the charging port 190 seals itself. The free end of the tube segment 164 is attached to a gas delivery device, such as one or more surgical instruments discussed above with regard to FIG. 1, by the Luer lock 168 or other appropriate connector. In another embodiment, the heater/humidifier 120 may be pre-charged with liquid, thus not requiring a charge prior to operation.

Once the insufflator 104 is activated, it receives gas from a gas supply cylinder and regulates the pressure and flow rate of the gas, both of which can be adjusted by the operator. The pressure and volumetric flow rate are controlled by adjusting controls (not shown) on the insufflator 104. Insufflator gas then flows through the tube segment 160 into the optional filter 110 where it is filtered, and then through tube segment 162 into the heater/humidifier 120. In the heater/humidifier 120, gas comes into contact with electrical heating element 134 and the humidifying liquid-retaining layer(s) 130-132 which are positioned within the flow path of the gas, as shown in FIG. 3. In one embodiment, the gas may pass through the humidifying liquid-retaining layer(s) 130-132. In another embodiment, some of the gas may pass through the humidifying liquid-retaining layer(s) 130-132, and/or some of the gas may pass over/under/around the humidifying liquid-retaining layer(s) 130-132. In chamber 128, insufflator gas may be simultaneously humidified and warmed to a desired physiological range by regulation of the heating element 134 and liquid content of the liquid-retaining layers 130-132 such that the temperature of gas exiting chamber 128 is within a desirable physiological temperature range (for example, 35° to 40° C., though any desired temperature range can be predetermined (or preselected), as is discussed above), and within a predetermined (or preselected) range of relative humidity. In one embodiment, the gas may be humidified so that it is within a range of relative humidity at the exit of the heater/humidifier for delivery into the first incision of the patient. It may also be within any of the following humidity ranges as the gas enters the patient through the exit of a delivery device. The relative humidity level may be above 40%, above 50%, above 60%, above 70%, above 75%, above 80%, above 85%, or above 90% relative humidity. In further embodiments, the range of relative humidity may be between 65-80%, between 70-85%, between 75-90%, between 80-95%, or any other suitable range. In some embodiments, the relative humidity may be between 95% and 100%. In one embodiment, if the apparatus is operated with the heater/humidifier 120 not charged with liquid either because the user forgot to manually charge it before initiating operation, or the apparatus was sold without a pre-charge of liquid (e.g., in a dry state), the relative humidity of the gas in the chamber of the heater/humidifier 120 will be detected to be below the predetermined threshold and the alarm will be activated, alerting the user that the heater/humidifier 120 requires charging of liquid. In one embodiment, the apparatus will automatically issue an alarm to alert a user to the need for charging the heater/humidifier 120 with liquid, thereby avoiding further delivery of unhumidified gas into a patient through, for example, the first incision.

With further reference to FIG. 6, the control module 140 monitors the relative humidity of the gas exiting the chamber and further regulates the temperature of the gas in the chamber 128. In particular, the microcontroller 142 generates a recharge signal when the relative humidity of the gas in the chamber drops below the predetermined relative humidity threshold, indicating that the liquid supply in the heater/humidifier 120 requires replenishing. An audible alarm is issued by the buzzer 147 and/or a visual alarm is issued by LED 148A to warn the medical attendant or user that the heater/humidifier 120 requires recharging. In one embodiment, the microcontroller 142 continues the alarm until the humidity in the chamber returns to a level above the predetermined relative humidity threshold, which will occur when the heater/humidifier 120 is recharged with liquid. Moreover, the microcontroller 142 will issue a second alarm, such as by energizing LED 148B, when the relative humidity level of gas in the heater/humidifier 120 drops below the critical relative humidity threshold, at which point electrical power to the heating element 134 is terminated. In a further embodiment, the microcontroller 142 controls the temperature of the gas by controlling electrical power supplied to the heating element 134.

In one embodiment, the apparatus of the present disclosure provides for control of the temperature and monitoring of the humidification of the gas, and of particular importance, generates an audible or visual alarm indicating that the heater/humidifier requires recharging of liquid to sustain and provide timed re-supply of liquid in order to maintain a flow of humidified/warmed gas. The alarm is maintained until the heater/humidifier is recharged and the humidity of the gas returns to a predetermined level. In a further embodiment, the apparatus disclosed herein is easily installed and prepared for use with a minimal amount of lines and tubes. The rechargeable feature of the heater/humidifier eliminates the need for an additional liquid supply tube connected to the heater/humidifier. If needed, the heater/humidifier may be recharged with liquid several times during a procedure.

In an additional embodiment, the power supply for the apparatus is derived from a standard AC wall outlet or power strip. Power strips are often provided on medical carts already used in the operating room environment. By using a power supply derived from a (normally) uninterrupted AC source, as opposed to the finite amount of power that can be supplied by a battery, accommodating surgical procedures, such as surgical method 10 of FIG. 1, that last longer than anticipated is not a concern. The control circuitry for the apparatus may be contained in an electrical housing that is relatively movable with respect to the remainder of the apparatus, and therefore can be placed in a non-interfering position in the operating room. For example, the electrical housing of the control module can be attached by tape or Velcro to the side of the insufflator or other stable structure in the operating room, and not encumber the remainder of the apparatus or affect parameter settings of the insufflator.

Although the embodiments in the disclosure have been described in detail, numerous changes, substitutions, variations, alterations, and modifications may be ascertained by those skilled in the art. It is intended that the present disclosure encompass all such changes, substitutions, variations, alterations, and modifications.

What is claimed is:

1. A surgical method for performing a coronary blood vessel bypass, comprising:
making a first incision in a limb of a patient;
receiving a gas from a source;
humidifying and warming the gas received from the source;
delivering the humidified and warmed gas into the first incision;
separating one or more blood vessel branches from a blood vessel using at least one surgical instrument inserted through a second incision in the limb of the patient;
removing a blood vessel segment from the patient through a third incision in the limb of the patient, wherein the blood vessel segment was in contact with the humidified and warmed gas delivered into the first incision prior to the removal; and
attaching at least a portion of the blood vessel segment to a coronary blood vessel.

2. The method of claim 1, wherein the humidifying and warming the gas received from the source comprises passing the gas through a chamber comprising a volume of a liquid.

3. The method of claim 2, wherein the liquid comprises sterile water or saline.

4. The method of claim 3, wherein the liquid further comprises one or more anesthetics.

5. The method of claim 3, wherein the liquid further comprises one or more anticoagulants.

6. The method of claim 2, further comprising injecting an additional amount of the liquid into the chamber.

7. The method of claim 2, wherein the volume of the liquid is contained in an absorbent material.

8. The method of claim 2, wherein the volume of the liquid is maintained between two or more membranes.

9. The method of claim 1, wherein the humidifying and warming the gas received from the source comprises increasing the humidity of the gas to a range of 80% through 95% relative humidity.

10. The method of claim 1, wherein the humidifying and warming the gas received from the source comprises increasing the humidity of the gas to above 40% relative humidity.

11. The method of claim 1, wherein the humidifying and warming the gas received from the source comprises increasing the humidity of the gas to above 60% relative humidity.

12. The method of claim 1, wherein the humidifying and warming the gas received from the source comprises increasing the humidity of the gas to above 80% relative humidity.

13. The method of claim 1, wherein the humidifying and warming the gas received from the source comprises increasing the humidity of the gas to above 90% relative humidity.

14. The method of claim 1, wherein the humidifying and warming the gas received from the source comprises increasing the humidity of the gas to above 95% relative humidity.

15. The method of claim 1, wherein the gas comprises carbon dioxide.

16. The method of claim 1, further comprising filtering the gas received from source.

17. The method of claim 1, wherein the blood vessel comprises a saphenous vein located in a leg of the patient.

18. The method of claim 1, wherein the blood vessel comprises a radial artery located in an arm of the patient.

19. The method of claim 1, further comprising monitoring a relative humidity of the humidified gas delivered into the incision of the patient.

20. The method of claim 1, wherein the first incision, the second incision, and the third incision are the same incision.

21. The method of claim 1, wherein one or more of the first incision, the second incision, and the third incision are different incisions.

22. A surgical method for performing a blood vessel bypass, comprising:
making a first incision in a patient;
receiving a gas from a source;
humidifying and warming the gas received from the source;
delivering the humidified and warmed gas into the first incision;
separating one or more blood vessel branches from a blood vessel using at least one surgical instrument inserted through a second incision in the patient;
removing a blood vessel segment from the patient through a third incision in the patient, wherein the blood vessel segment was in contact with the humidified and warmed gas delivered into the first incision prior to the removal; and
attaching at least a portion of the blood vessel segment to a second blood vessel.

23. A surgical method, comprising:
making a first incision in a patient;
receiving a gas from a source;
humidifying and warming the gas received from the source;
delivering the humidified and warmed gas into the first incision;
separating one or more blood vessel branches from a blood vessel using at least one surgical instrument inserted through a second incision in the patient; and
removing a blood vessel segment from the patient through a third incision in the patient, wherein the blood vessel segment was in contact with the humidified and warmed gas delivered into the first incision prior to the removal.
